(19) United States
(12) Patent Application Publication
(10) Pub. No.: US 2024/0034952 A1
(43) Pub. Date: Feb. 1, 2024

(54) MEDICAL DEVICE, FUNCTIONAL PART FOR A MEDICAL DEVICE AND METHOD FOR STERILIZING AND/OR PRODUCING STERILIZATION RESISTANCE OF A MEDICAL DEVICE OR FUNCTIONAL PART

(71) Applicant: B. Braun Melsungen AG, Melsungen (DE)

(72) Inventors: Manuel Arnaldi, Ruederswil (CH); Liesa Glaess, Schuepfheim (CH)

(21) Appl. No.: 18/360,603

(22) Filed: Jul. 27, 2023

(30) Foreign Application Priority Data
Jul. 28, 2022 (DE) 10 2022 207 782.2

(51) Int. Cl. C10M 107/50; A61M 5/142; A61L 2/08; C10M 119/22; C10M 113/12; C10M 123/04

(52) U.S. Cl. CPC C10M 107/50; A61M 5/142; A61L 2/081; A61L 2/087; A61L 2/082; C10M 119/22; C10M 113/12; C10M 123/04; A61L 2202/24; C10N 2040/50

(57) ABSTRACT

A medical device, an elastically deformable functional part for a medical device, and a method for sterilizing and/or for establishing sterilization resistance of a medical device or an elastically deformable functional part. The elastically deformable functional part has an openable slit arrangement that widens or opens upon elastic deformation of the elastically deformable functional part and recloses upon cessation of the elastic deformation. The slit wall faces of the slit arrangement are coated with a composition that includes a silicone oil and a thickener.

### CROSS-REFERENCE TO RELATED APPLICATION(S)

This application claims priority under 35 U.S.C. § 119 to German Application No. 10 2022 207 782.2, filed on Jul. 28, 2022, the content of which is incorporated by reference herein in its entirety.

### FIELD

The present disclosure relates to a medical device comprising an elastically deformable functional part having an openable slit arrangement, to a corresponding functional part for a medical device and to a method for sterilizing and/or for establishing sterilization resistance of such a medical device or functional part.

### BACKGROUND

Gamma irradiation is a common sterilization method for medical devices. If the medical devices comprise a radiation-crosslinkable material, such as silicone elastomers, these materials will react with crosslinking reactions when exposed to high-energy radiation. In the case of slit valves, this can lead to an undesirable closing up of the slits, which is also referred to as healing.

In order to prevent such healing of slits, additives are usually added. One disadvantage is that such additivation generally leads to opaque materials. However, for many applications, for example connection devices in the field of medical infusion therapy, the maintenance of transparency of individual functional parts, such as valve bodies, is absolutely necessary for the intended use. Another problem is that the addition of solid additives can cause fundamental changes in material properties.

In addition to solid additives, liquid additives are also used as separating agents. However, they do not have sufficient wettability and, in particular, steadfastness in order to be able to perform their function lastingly. Radiation sterilization, ageing processes and transport processes generally involve elevated temperatures, as a result of which the separation effect of liquid separating agents over time is frequently found to be too low.

### SUMMARY

It is therefore an object of the present disclosure to provide a medical device and a functional part for a medical device that partially or completely prevents disadvantages known in connection with medical devices or functional parts of the type in question, in particular closing up of slits and/or loss of transparency. It is a further object of the present disclosure to provide a method for sterilizing and/or for establishing sterilization resistance of a medical device or functional part for a medical device that partially or completely circumvents the disadvantages mentioned at the start.

According to a first aspect, the present disclosure relates to a medical device.

The medical device comprises an elastically deformable functional part having an openable slit arrangement, wherein the slit arrangement widens, opens or opens out upon elastic deformation of the elastically deformable functional part and recloses upon cessation of the elastic deformation. Preferably, the manner in which the slit arrangement widens upon the elastic deformation of the elastically deformable functional part or the slit arrangement opens upon the elastic deformation of the elastically deformable functional part is such that said slit arrangement allows a flow of a fluid, in particular a liquid, preferably medical solution, or a gas, through the elastically deformable functional part. Elastic deformation of the elastically deformable functional part can occur, for example, when connecting the medical device to an infusion syringe, connectors, in particular Luer slip connections and/or Luer lock connections according to ISO80369-7, a flexible tube or flexible tube system or the like, especially of a medical infusion system.

The medical device is particularly distinguished by the fact that slit wall faces of the slit arrangement are provided or coated, in particular wetted, with a composition comprising an oil and a thickener, preferably a biocompatible oil and a biocompatible thickener, or consisting of an oil and a thickener, preferably a biocompatible oil and a biocompatible thickener.

Preferably, the slit wall faces, in particular only the slit wall faces, of the slit arrangement are provided or coated with the composition at least partially, in particular only partially or completely, i.e. across the entire surface or continuously.

In a closed position of the slit arrangement, the slit wall faces are preferably arranged or positioned next to one another at least sectionally, in particular only sectionally or, this being preferred, continuously, in particular along a fluid passage axis, in particular central longitudinal axis, of the elastically deformable functional part. In an open position of the slit arrangement, the slit wall faces or slit walls are preferably pushed apart, for example by an infusion syringe, connectors, in particular Luer slip connections and/or Luer lock connections according to ISO80369-7, a flexible tube or a flexible tube system or the like, especially of a medical infusion system.

In the context of the present disclosure, the expression “functional part” is to be understood to mean a component or a component part or a construction element of the medical device.

In the context of the present disclosure, the expression “slit wall faces” is to be understood to mean faces or surfaces of walls that form or define a slit or are involved in the formation of a slit.

In the context of the present disclosure, the expression “separating agent” is to be understood to mean a substance or composition capable of reducing or (completely) avoiding closing up of slits, in particular that induced by radiation.

In the context of the present disclosure, the expression “biocompatible oil” or “biocompatible thickener” is to be understood to mean an oil or thickener which can be used in the field of medicine, in particular in medical fields with direct or indirect patient contact, without health concerns or risks.

In the context of the present disclosure, the expression “thickener” is to be understood to mean a substance which increases the consistency or viscosity of the composition.

The present disclosure is based on the surprising finding that the composition envisaged by the present disclosure is suitable as separating agent for preventing healing phenomena, i.e. closing up of slits, in elastically deformable functional parts and medical devices comprising such functional parts. Owing to its constituents, the composition according to the present disclosure is advantageously distinguished by sufficient wettability and, in particular, steadfastness. As a result, it can in turn exhibit its separation function in the long term or lastingly. Upon exposure to radiation, in particular gamma radiation, beta radiation, X-radiation or electron beam radiation (E-beam radiation), better protection against radiation-induced crosslinking phenomena is thus achievable. It is especially advantageous that the present disclosure makes it possible to carry out radiation sterilization, in particular gamma radiation sterilization, with a radiation dose of at least 18 kGy, in particular at least 32 kGy, in particular at least 40 kGy, without (appreciable) closing up of slits. Furthermore, it is advantageous that an increase in ageing resistance is achievable by the present disclosure.

In one embodiment of the present disclosure, the oil is selected from the group consisting of silicone oil, alkylated naphthalene (AN), chlorotrifluoroethylene (CTFE), ester oil, mineral oil, in particular synthetic mineral oil, multiple alkylated cyclopentane (MAC), polyalphaolefin (PAO), polyphenyl ether (PPE), polyglycol oil (PG), perfluorinated polyether oil (PFPE), triglycerides and mixtures of at least two of the aforementioned oils.

The aforementioned oils have in particular the advantage that they are of particularly low reactivity with respect to radiation sterilization, thereby additionally optimizing the separation effect of the composition according to the present disclosure.

In a further embodiment of the present disclosure, the oil is a silicone oil. In particular, the silicone oil may be a fluorinated silicone oil, in particular partially fluorinated or perfluorinated silicone oil.

Particularly preferably, the oil is a perfluorinated silicone oil. In this respect, it has been found advantageous that a silicone oil having a relatively high fluorine content reacts less to radiation, in particular gamma radiation, beta radiation, X-radiation or electron beam radiation (E-beam radiation), and better protection against radiation-induced crosslinking phenomena is thus achievable.

In a further embodiment of the present disclosure, the oil has a proportion of 0.1% by weight to 99.9% by weight, in particular 10% by weight to 90% by weight, preferably 20% by weight to 80% by weight, based on the total weight of the composition.

The thickener is preferably a solid.

In a further embodiment of the present disclosure, the thickener is a metal soap, in particular light metal soap, particularly preferably alkali metal soap and/or an alkaline earth metal soap.

In the context of the present disclosure, the expression “light metal” is to be understood to mean aluminium, alkali metals, in particular lithium, sodium, potassium, rubidium, caesium or francium, alkaline earth metals, in particular beryllium, magnesium, calcium, strontium or barium, or mixtures of at least two of the aforementioned metals.

In the context of the present disclosure, the expression “metal soap” is to be understood to mean a metal salt of a fatty acid, a so-called single soap, or a metal salt of a fatty acid mixture, a so-called complex soap. The fatty acid may in particular be selected from the group consisting of formic acid, acetic acid, propionic acid, butyric acid, valeric acid, capronic acid, enanthic acid, caprylic acid, pelargonic acid, capric acid, lauric acid, myristic acid, palmitic acid, margaric acid, stearic acid, arachidic acid, behenic acid, lignoceric acid, cerotic acid, montanic acid, melissic acid, lacceroic acid, geddic acid, undecylenic acid, myristoleic acid, palmitoleic acid, margaroleic acid, petroselinic acid, oleic acid (OA), elaidic acid, vaccenic acid, gadoleic acid, gondoic acid, cetoleic acid, erucic acid, nervonic acid, linoleic acid (LA), alpha-linolenic acid, gamma-linolenic acid, calendic acid, punicic acid, alpha-eleostearic acid, beta-eleostearic acid, stearidonic acid, arachidonic acid, eicosapentaenoic acid, docosadienoic acid, docosatetraenoic acid, docosapentaenoic acid, docosahexaenoic acid, tetracosahexaenoic acid and mixtures of at least two of the aforementioned fatty acids.

In particular, the metal soap may be selected from the group consisting of single soaps, in particular aluminium soap, barium soap, calcium soap, lithium soap or sodium soap, and complex soaps, in particular aluminium complex soap, barium complex soap, calcium complex soap, lithium complex soap or sodium complex soap, and mixtures of at least two of the aforementioned metal soaps.

In a further embodiment of the present disclosure, the thickener is selected from the group consisting of polytetrafluoroethylene (PTFE), inorganic thickener, in particular bentonite, polyurea, silica, glucose, ascorbic acid, starch and mixtures of at least two of the aforementioned thickeners.

In a further embodiment of the present disclosure, the thickener is polytetrafluoroethylene (PTFE) or silica or a mixture comprising or consisting of polytetrafluoroethylene (PTFE) and silica.

In a further embodiment of the present disclosure, the thickener has a proportion of by weight to 99.9% by weight, in particular 10% by weight to 90% by weight, preferably 20% by weight to 80% by weight, based on the total weight of the composition.

The aforementioned thickeners have been found particularly advantageous for giving the composition according to the present disclosure a certain wettability and, in particular, steadfastness, as a result of which the separation effect of the composition can be exhibited particularly well.

In a further embodiment of the present disclosure, the composition further comprises a solid, in particular in the form of a powder, which is not a thickener. The solid is preferably selected from the group consisting of salts, saccharides, vitamins and mixtures of at least two of the aforementioned solids.

In a further embodiment of the present disclosure, the solid has a proportion of 0.01% by weight to 99.99% by weight, in particular 1% by weight to 99% by weight, preferably 10% by weight to 90% by weight, based on the total weight of the composition.

The solid may have a particle size or an average particle diameter of <(in words: smaller than) 400 μm, in particular 1 μm to 50 μm, preferably 0.01 μm to 10 μm.

The salts may in particular be an alkali metal salt, preferably selected from the group consisting of alkali metal chloride, alkali metal bromide, alkali metal iodide and mixtures of at least two of the aforementioned alkali metal salts.

The alkali metal chloride may in particular be selected from the group consisting of lithium chloride, sodium chloride, potassium chloride and mixtures of at least two of the aforementioned alkali metal chlorides.

The alkali metal bromide may in particular be selected from the group consisting of lithium bromide, sodium bromide, potassium bromide and mixtures of at least two of the aforementioned alkali metal bromides.

The alkali metal iodide may in particular be selected from the group consisting of lithium iodide, sodium iodide, potassium iodide and mixtures of at least two of the aforementioned alkali metal iodides.

Particularly preferably, the solid is sodium chloride.

When using one or more of the aforementioned salts, in particular sodium chloride, the advantages of the present disclosure can be additionally improved.

The saccharides may in principle be monosaccharides, oligosaccharides, such as disaccharides, or polysaccharides, or a mixture of at least two of the aforementioned saccharides.

Preferably, the saccharides are glucose.

By using saccharides, in particular glucose, the advantages of the present disclosure can be (likewise) additionally optimized.

The vitamins may in particular be ascorbic acid, preferably L-(+)-ascorbic acid, i.e. vitamin C. The advantages of the present disclosure can be (also) additionally optimized when using ascorbic acid, preferably L-(+)-ascorbic acid or vitamin C.

In a further embodiment of the present disclosure, the composition is a viscous or pasty composition. Such a composition shows the advantages of the present disclosure particularly well. In particular, the disadvantages described at the start in connection with liquid additives can be avoided thereby particularly well. Preferably, the composition is in the form of a lubricating grease composition.

More preferably, the composition or the oil of the composition has a viscosity of 100 mm²/s to 1 000 000 mm²/s, in particular 200 mm²/s to 12 500 mm²/s, preferably 300 mm²/s to 2000 mm²/s. The viscosity is preferably determined at 20° C. or 40° C.

In a further embodiment of the present disclosure, the functional part is at least sectionally transparent, i.e. light-transmissive, in particular only sectionally or completely transparent. In the context of the present disclosure, the expression “transparent” or “light-transmissive” is to be understood to mean the ability of matter to allow electromagnetic waves having a wavelength of 380 nm to 780 nm to pass through (transmission). In this respect, the present disclosure is based on the further surprising finding that, in the case of a transparent functional part, the composition envisaged by the present disclosure does not lead to an opaque change in the functional part. Instead, the transparency of the elastically deformable functional part is advantageously maintained.

In a further embodiment of the present disclosure, the elastically deformable functional part comprises an elastically deformable material, in particular a transparent, i.e. light-transmissive, elastically deformable material, or the elastically deformable functional part, in particular as one piece, is made of an elastically deformable material, in particular transparent, i.e. light-transmissive, elastically deformable material.

The elastically deformable material is preferably an elastomer and/or thermoplastic elastomer.

The elastomer may in particular be selected from the group consisting of silicone elastomers such as silicone rubber, acrylonitrile-butadiene rubber, hydrogenated acrylonitrile-butadiene rubber, styrene-butadiene rubber and mixtures of at least two of the aforementioned elastomers. Alternatively or in combination, the elastomer may be another polymer which is crosslinking-capable or in which crosslinking reactions predominate.

In a further embodiment of the present disclosure, the slit wall faces, in particular only the slit wall faces, of the slit arrangement are coated, in particular wetted, with the composition at least partially, in particular only partially or completely, i.e. continuously or across the entire surface. Preferably, the elastically deformable functional part, apart from the slit wall faces of the slit arrangement, is free of the composition. In particular, the composition is not present, in particular not dispersed or distributed, within the elastically deformable functional part. Instead, the composition is preferably exclusively present on the slit wall faces of the slit arrangement. This can advantageously additionally reduce the risk that material properties, such as in particular transparency and/or mechanical properties, of the functional part change.

Furthermore, the slit wall faces, in particular only the slit wall faces, of the slit arrangement may be provided or coated with 0.01 mg to 2 mg, in particular 0.8 mg to 1.2 mg, preferably 1 mg, of the composition at least partially, in particular only partially or completely, i.e. continuously or across the entire surface.

Furthermore, the slit wall faces, in particular only the slit wall faces, of the slit arrangement may be provided or coated with the composition at least partially, in particular only partially or completely, i.e. continuously or across the entire surface, at a thickness of 0.01 μm to 800 μm, in particular 0.1 μm to 400 μm, preferably 1 μm to 20 μm.

In a further embodiment of the present disclosure, the slit arrangement is in the form of a longitudinal slit arrangement, i.e. linear slit arrangement.

Alternatively, the slit arrangement may deviate from this, for example may be Y-shaped or cross-shaped, i.e. may be in the form of a so-called cross-slit arrangement.

In a further embodiment of the present disclosure, the elastically deformable functional part is in the form of a valve body.

In a further embodiment of the present disclosure, the elastically deformable functional part, in particular the valve body, has a head region, in particular a head region in lid form. The slit arrangement is preferably formed along a transverse extent, i.e. transverse to a longitudinal extent, of the head region. Particularly preferably, the slit arrangement extends, in particular centrally, along a longitudinal axis or fluid passage axis, in particular the central longitudinal axis, through the head region. Preferably, a casing of the elastically deformable functional part adjoins the head region, in particular directly. If the elastically deformable functional part is in the form of a valve body, this being preferred according to the present disclosure, a valve casing adjoins the head region, in particular directly. The valve casing preferably has a radial cross-section which preferably has a larger longitudinal extent than transverse extent. The radial cross-section of the valve casing is based on a longitudinal axis or fluid passage axis of the valve body, which preferably corresponds to a central longitudinal axis of the valve body. The head region and/or casing, for example only the head region or only the casing, of the elastically deformable functional part, in particular valve body, may be transparent, i.e. may comprise a transparent, elastically deformable material, preferably an elastomer, in particular silicone elastomer, and/or thermoplastic elastomer, or may be made of such a material. Regarding further suitable materials, reference may be made to the previous description.

Preferably, the elastically deformable functional part is used for a connection device of a medical infusion system. A relevant connection device is known, for example, from EP 3 216 486 A1. The features and advantages described there in relation to the connection device are incorporated in the present description by express reference.

In a further embodiment of the present disclosure, the medical device is a medical device for infusion therapy. Especially preferably, the medical device is a connection device, especially of a medical infusion system or for a medical infusion system. The medical device, in particular the connection device, may be, for example, in the form of a one-way valve, two-way valve, three-way valve or four-way valve or in the form of a valve manifold, i.e. in the form of a unit or system having or composed of one-way valves arranged or connected one after the other, i.e. in series. Preferably, the medical device, in particular the connection device, is in the form of a two-way valve, three-way valve or four-way valve or in the form of a valve manifold.

Preferably, the medical device is in sterilized form, in particular radiation-sterilized form, preferably gamma radiation-sterilized, beta radiation-sterilized, x-radiation-sterilized or electron beam-sterilized form. Preferably, the medical device is sterilized by irradiation, preferably gamma irradiation, beta irradiation, X-ray irradiation or electron beam irradiation (E-beam irradiation), in particular with a radiation dose of 18 kGy to 100 kGy, preferably 18 kGy to 40 kGy, in particular 18 kGy to 32 kGy. Especially preferably, the medical device may be sterilized with a radiation dose of 32 kGy, preferably 40 kGy, to 100 kGy.

Alternatively, the medical device may be in non-sterilized form.

According to a second aspect, the present disclosure relates to an elastically deformable functional part for a medical device.

The elastically deformable functional part comprises an openable slit arrangement which widens, opens or opens out upon elastic deformation of the elastically deformable functional part and recloses upon cessation of the elastic deformation. Slit wall faces of the slit arrangement are provided or coated, in particular wetted, with a composition comprising an oil, preferably biocompatible oil, and a thickener, preferably biocompatible thickener, or consisting of an oil, preferably biocompatible oil, and a thickener, preferably biocompatible thickener.

Regarding further features and advantages of the elastically deformable functional part, in particular the slit arrangement and the composition, and of the medical device, full reference is made to the corresponding statements made under the first aspect of the present disclosure. The features and advantages described there in this respect also apply mutatis mutandis to the elastically deformable functional part according to the second aspect of the present disclosure.

According to a third aspect, the present disclosure relates to a method for sterilizing and/or for establishing sterilization resistance of a medical device according to the first aspect of the present disclosure or of an elastically deformable functional part according to the second aspect of the present disclosure. The method comprises, preferably in chronological order, the following steps:
a) coating or loading, in particular wetting, the slit wall faces of the slit arrangement with the composition comprising an oil and a thickener, preferably a biocompatible oil and a biocompatible thickener, or consisting of an oil, preferably biocompatible oil, and a thickener, preferably biocompatible thickener, and
b) sterilizing the medical device or elastically deformable functional part, in particular by exposure to radiation, preferably gamma radiation, X-radiation, beta radiation or electron beam radiation (E-beam radiation).

The method according to the third aspect of the present disclosure is preferably distinguished by the fact that step b) is carried out after step a) has been carried out.

The slit wall faces of the slit arrangement may be coated or loaded, in particular wetted, with the composition only partially or completely, i.e. continuously or across the entire surface.

In a further embodiment of the present disclosure, the slit wall faces, in particular only the slit wall faces, of the slit arrangement are provided or coated, in particular wetted, with 0.01 mg to 2 mg, in particular 0.8 mg to 1.2 mg, preferably 1 mg, of the composition at least partially, in particular only partially or completely, i.e. continuously or across the entire surface.

Furthermore, step b) may be carried out with a radiation dose of 18 kGy to 100 kGy, preferably 18 kGy to 40 kGy, in particular 18 kGy to 32 kGy. In particular, step b) may be carried out with a radiation dose of 32 kGy, preferably 40 kGy, to 100 kGy.

Regarding further features and advantages of the method, full reference is made to the corresponding statements made under the first and second aspects of the present disclosure. The features and advantages described there especially in relation to the elastically deformable functional part and to the medical device also apply mutatis mutandis to the method according to the third aspect of the present disclosure.

Further features and advantages of the present disclosure will become apparent from the following description of preferred embodiments with reference to figures and figure descriptions. Here, individual features of the present disclosure can be realized singly or plurally in combination with one another. The preferred embodiments merely serve to elucidate the present disclosure, which is not to be limited thereto in any way.

### BRIEF DESCRIPTION OF THE DRAWINGS

FIG. 1 shows a plan view of one embodiment of a medical device according to the present disclosure in the form of a connection device;

FIG. 2 shows a section through the medical device according to FIG. 1 along the section line II-II in FIG. 1;

FIG. 3 shows a view from below of a functional part of the medical device according to FIG. 2 in the form of a valve body;

FIG. 4 shows a longitudinal section through the valve body according to FIG. 3 along the section line IV-IV in FIG. 3;

FIG. 5 shows a further longitudinal section through the valve bodies along the section line V-V in FIG. 3;

FIG. 6 shows a plan view of the valve body according to FIGS. 3 to 5;

FIGS. 7 to 9 show sectional views of different steps during connection of a component to the medical device according to FIG. 2; and

FIGS. 10a and 10b show one embodiment of a method according to the present disclosure.

### DETAILED DESCRIPTION

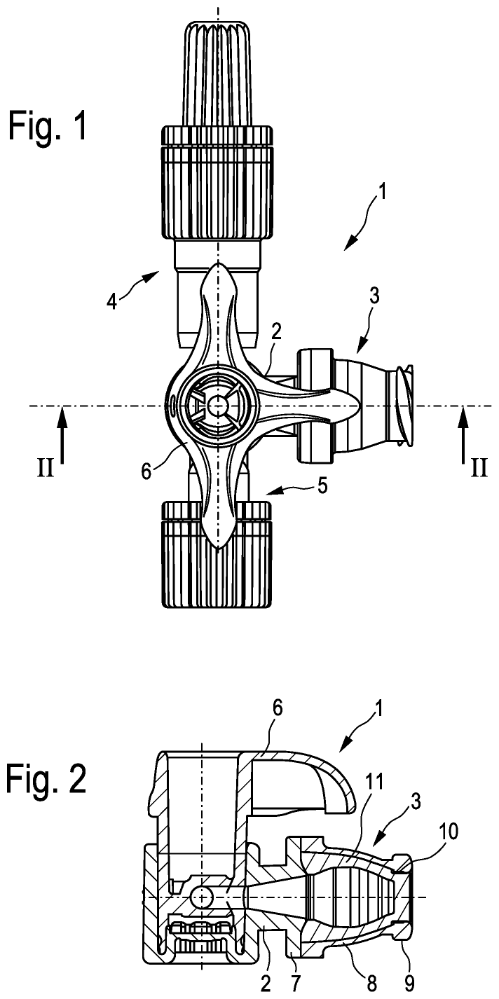

FIG. 1 shows schematically one embodiment of a medical device according to the present disclosure in the form of a connection device 1 of a medical infusion system. The connection device 1 may, for example, be in the form of a three-way valve, as shown in FIG. 1. It will be appreciated, however, that the connection device 1 may deviate from this, in particular may be in the form of a one-way or two-way valve or in the form of a valve manifold, i.e. in the form of a unit or system having or composed of one-way valves connected in series, i.e. one after the other.

The connection device 1 comprises a housing 2, a connection nozzle 3 and two connection regions 4, 5. Rotatably mounted in the housing 2 is an actuator 6. Further provided in the housing 2 are a total of three connection channels which are blocked or connected to each other depending on the position of the actuator 6. One connection channel of the housing 2 leads to the connection nozzle 3. A further connection channel arranged at a right angle leads to the connection region 4 and, opposite to this, a third connection channel leads to the connection region 5. One of the two connection regions 4, 5 is intended for the connection of a patient line. The other connection region 4, 5 is used to connect a connection line to a fluid container. The connection nozzle 3 is preferably provided for temporary connection of a component of the medical infusion system, such as in particular a syringe, in order to supply the patient line with, for example, (additional) medicaments or the like. Additionally or alternatively, the connection nozzle 3 may be provided for connection, in particular continuous connection (permanent connection), of an additional line.

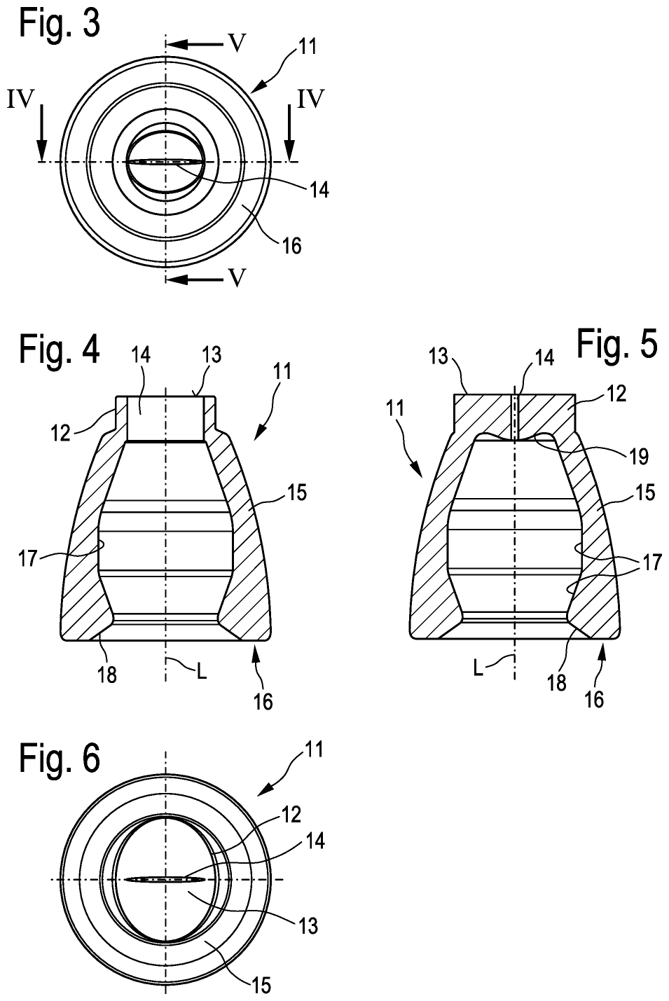

The connection nozzle 3 will be more particularly elucidated with reference to FIGS. 3 to 5 that follow.

The connection nozzle 3 may have a dimensionally stable cap 8 which, at its end face that faces the housing 2, is connected, preferably fixedly connected, to a dimensionally stable base section 7 of the housing 2. The cap 8 is sleeve-shaped and has, on the side facing the base section 7, a thickened edge region which is fixedly connected to the base section 7, for example by welding. The base section 7 is dish-shaped and protrudes radially outwards relative to a central longitudinal axis L of the connection nozzle 3. The base section 7 surrounds a channel section which conically tapers or non-conically tapers towards an interior of the housing 2. Alternatively, the channel section may be cylindrical or non-cylindrical.

At its end region that is remote from the base section 7, the cap 8 is provided with a passage 10 which is closable by a valve body 11 of the connection device 1 that will be described in greater detail below. The passage 10 is enclosed by a thickened edge region which is provided with connection profiling 9 in the form of Luer-lock profiling.

The valve body 11 is preferably cup-shaped or bell-shaped and in particular made of an elastically deformable material as a single piece. The elastically deformable material is preferably an elastomer or thermoplastic elastomer. Particularly preferably, the elastically deformable material is a silicone elastomer, such as silicone rubber. Preferably, the elastically deformable material is moreover transparent, i.e. light-transmissive.

The valve body 11 preferably has an outer contour which, in an unloaded, i.e. non-deformed, initial state, is in flush and planar contact with the inner contour of the cap 8 over a total height of the cap 8. The valve body 11 has a head region 12, in particular a head region in lid form. The head region 12 may have a rotationally symmetrical, in particular circular, or, as depicted, a rotationally unsymmetrical, in particular oval, main face (see in particular FIGS. 3 and 6). The valve body 11 further comprises a valve casing 15. The valve casing 15 adjoins the head region 12, preferably directly. Preferably, the valve casing 15 is provided with a base ring 16 at a lower end-face region. The head region 12 is provided with a slit arrangement 14. A surface 13 of the head region 12 is preferably smooth and flat.

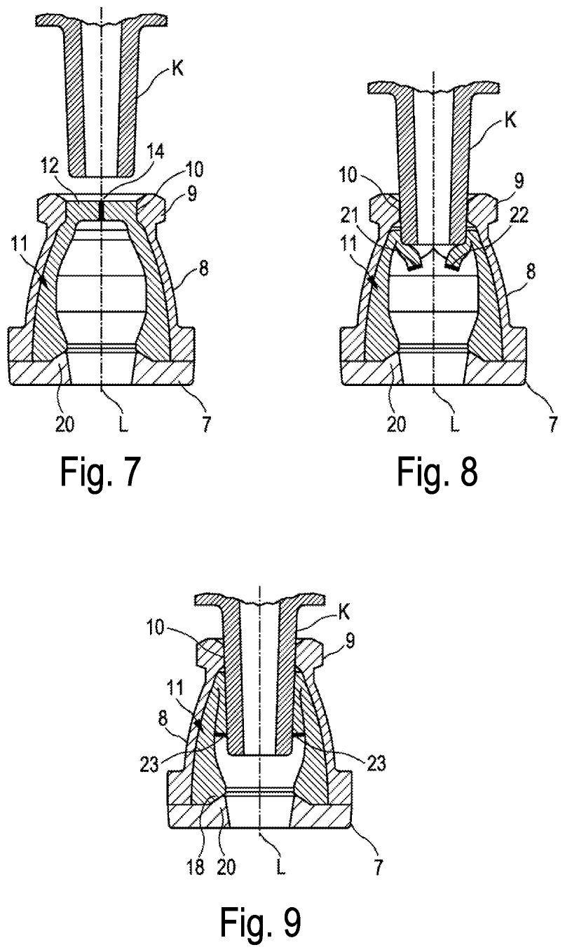

From FIG. 7, it can be seen that, in an unloaded initial state of the valve body 11, the surface 13 of the head region 12 can be flush in the cap 8 with a marginal edge of the passage From said marginal edge of the passage 10, an oblique chamfer extends outwards up to an end face of the edge region of the cap 8 that defines the passage 10. Consequently, in the unloaded initial state of the valve body 11, the end face of the edge region of the cap 8 including the surface 13 of the head region 12 of the valve body 11 can be cleaned and disinfected in a simple manner, for example by means of a disinfectant cloth or the like, by medical personnel.

The valve casing 15 of the valve body 11 is preferably rotationally symmetrical relative to the central longitudinal axis L. Preferably, the valve casing 15 has a wall which thickens starting from the head region 12 up to the base ring 16. Said thickening may occur discontinuously and non-linearly, as can be seen from the two visible edges shown. The edges are preferably annularly circumferential. The valve casing 15 may have a first wall section of constant thickness that adjoins the head region 12 and expands like a truncated cone. Adjoining said first wall section, in the direction of the base ring 16, may be a second wall section, the inner wall of which runs cylindrically and coaxially in relation to the central longitudinal axis L and the outer wall of which runs bulgingly further outwards towards the base ring 16. Adjoining said wall section, in particular central wall section, may be a base-side wall section. Preferably, the base-side wall section comprises the base ring 16. Preferably, in this region, the inner wall tapers towards the base section starting from the cylindrical central region, thereby yielding an inner wall section which conically tapers downwards.

The inner wall may conically expand again towards the end face of the base ring 16 to form a contact surface 18. Consequently, an egg-shaped or O-shaped inner contour 17 can result over the height of the valve casing 15 (see FIGS. 4 and 5).

An inner face of the head region 12 pointing into the interior of the valve casing 15 may be a dome-shaped contour 19, as shown in FIG. 5.

The passage 10 of the cap 8 may be rotationally symmetrical or rotationally unsymmetrical. In the case of a rotationally unsymmetrical head region 12, the passage 10 is preferably also rotationally unsymmetrical in a complementary manner.

As can be seen from FIGS. 2 and 7 to 9, the conical contact surface 18 of the base ring 16 of the valve body 11 is assigned a complementarily conical support face 20 in the region of the base section 7, as a result of which the valve body 11 is planarly supported on the base section 7 in the region of the base ring 16 over its entire radial width.

The slit arrangement 14 is oriented transversely to a longitudinal extent of the head region 12, as can be seen from FIGS. 4 to 6. The slit arrangement 14 preferably extends centrally along the central longitudinal axis L through the head region 12. The slit arrangement 14 has two slit wall faces 21, 22 which close, preferably tightly, the slit arrangement 14 in an unloaded initial state of the valve body 11.

The slit wall faces 21, 22 of the slit arrangement 14 are provided with a composition 23. The slit wall faces 21, 22 may be provided, in particular coated, with the composition 23 only partially or completely, i.e. across the entire surface.

Preferably, the composition 23 is present only on the slit wall faces 21, 22 of the slit arrangement 14. In other words, especially the head region 12 remaining and the valve casing are preferably composition-free, i.e. free of the composition.

The composition 23 can advantageously prevent radiation-induced crosslinking of the elastically deformable material of the valve body 11, for example during gamma sterilization, beta sterilization, X-ray sterilization or electron beam sterilization (E-beam sterilization) of the connection device 1. This can significantly reduce or even completely avoid the risk of closing up of the slit arrangement 14.

The composition 23 comprises an oil and a thickener, preferably a biocompatible oil and a biocompatible thickener, or consists of an oil and a thickener, preferably a biocompatible oil and a biocompatible thickener. The oil is preferably a silicone oil, in particular a partially fluorinated or perfluorinated silicone oil. Alternatively, the oil may also be a fluorine-free silicone oil. The thickener is preferably polytetrafluoroethylene (PTFE) and/or silica. Preferably, the composition is in the form of a viscous or pasty composition. Particularly preferably, the composition is a lubricating grease composition.

The aforementioned composition has been found to be particularly suitable with respect to avoiding closing up or healing of the slit arrangement 14 during radiation sterilization, such as in particular gamma radiation sterilization. In particular, a further advantage of these substances is that they do not affect any transparency of the valve body 11. In addition, the aforementioned substances can advantageously contribute to an increase in the ageing resistance of the valve body 11.

Once a tip of a component K to be connected to the connection nozzle 3 is brought from the outside to the connection nozzle 3, the tip comes into planar contact with the outer surface of the head region 12 and pushes the head region 12 into the interior of the cap 8. In this case, the slit arrangement 14 widens, and elastically deformed sections of the head region 12 are placed against the outside of the tip of the component K on further penetration of said tip. When the tip is disconnected and consequently removed outwardly, the head region 12 returns to the initial position according to FIG. 7.

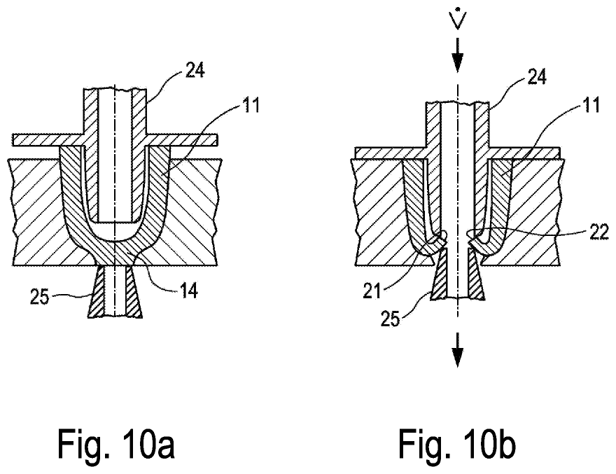

FIGS. 10a and b show schematically one embodiment of a method according to the present disclosure.

The method first comprises coating the slit wall faces 21, 22 of the slit arrangement 14 with the composition. To this end, a valve body 11 is placed in a receptacle or directly on a nozzle 24. At the same time, the nozzle 24 can be brought closer using a hold-down device and/or a compression overlay until the valve body 11 is compressed. Furthermore, the valve body 11, as shown in FIGS. 10a and b, can be pressed against a Luer-like or cylindrical component 25, bringing about an at least partial opening of the valve body 11. In this case, the component 25, as shown in FIG. 10b, can enter somewhat.

Preferably, the nozzle 24 has a diameter of 0.1 mm to 10 mm, preferably 0.9 mm to 2 mm. The aforementioned nozzle diameters are particularly well suited for coating the slit wall faces 21, 22 of the slit arrangement 14 with the composition and for preventing closing up of the slits by the composition according to the present disclosure. Furthermore, the aforementioned nozzle diameters can advantageously prevent segregation of the composition, i.e. separation of the composition into oil and thickener.

Furthermore, the nozzle 24 may be elliptic or in the form of a slotted nozzle. If necessary, an orientation in relation to the slit wall faces 21, 22 of the slit arrangement 14 may be expedient.

Alternatively or in combination, the slit wall faces 21, 22 of the slit arrangement 14 may be subjected to spreading or smearing of the composition with the aid of a spreading element, in particular a doctor blade.

Alternatively or in combination, the composition may be applied by an air stream or mist, in particular a damp or dry mist. Preferably, the air stream or mist is conducted through the partially or fully open valve body 11 in such a way that the air stream or mist floods the slit wall faces 21, 22 of the slit arrangement 14 with the composition. The composition is transported by the air stream or mist in the direction of the arrow (see FIG. 10b). The air or mist pressure for transporting the composition to the slit wall faces 21, 22 of the slit arrangement 14 may be selected from 0.1 bar to 7 bar, preferably 0.5 bar to 3 bar. Furthermore, a volume flow time of 1 ms to 5 s, in particular 50 ms to 1 s, may be selected.

Preferably, the valve body 11 is compressed and thus opened in such a way that the inner lumen of the valve body 11 is not coated.

1. A medical device comprising an elastically deformable functional part having a slit arrangement that is openable, wherein the slit arrangement widens or opens upon an elastic deformation of the elastically deformable functional part and recloses upon cessation of the elastic deformation, wherein slit wall faces of the slit arrangement are coated with a composition, wherein the composition comprises a silicone oil and a thickener.

2. The medical device according to claim 1, wherein the silicone oil is a fluorinated silicone oil.

3. The medical device according to claim 1, wherein the silicone oil has a proportion of 0.1% by weight to 99.9% by weight based on the total weight of the composition.

4. The medical device according to claim 1, wherein the thickener is selected from the group consisting of: aluminium soap, aluminium complex soap, barium soap, barium complex soap, calcium soap, calcium complex soap, lithium soap, lithium complex soap, sodium soap, sodium complex soap, polytetrafluoroethylene (PTFE), inorganic thickener, in particular bentonite, polyurea, silica, glucose, ascorbic acid, starch and mixtures of at least two of the aforementioned thickeners.

5. The medical device according to claim 1, wherein the thickener is polytetrafluoroethylene (PTFE), silica or a mixture of polytetrafluoroethylene (PTFE) and silica.

6. The medical device according to claim 1, wherein the thickener has a proportion of 0.1% by weight to 99.9% by weight based on the total weight of the composition.

7. The medical device according to claim 1, wherein the composition further comprises a solid which is not a thickener.

8. The medical device according to claim 7, wherein the solid is selected from the group consisting of salts, saccharides, vitamins and mixtures of at least two of the aforementioned solids.

9. The medical device according to claim 7, wherein the solid has a proportion of 0.01% by weight to 99.99% by weight based on the total weight of the composition.

10. The medical device according to claim 1, wherein the composition is a viscous or pasty composition.

11. The medical device according to claim 1, wherein the elastically deformable functional part is at least sectionally transparent or light-transmissive.

12. The medical device according to claim 1, wherein the elastically deformable functional part is made of an elastically deformable material.

13. The medical device according to claim 12, wherein the elastically deformable material is an elastomer.

14. The medical device according to claim 1, wherein the slit arrangement is formed as a longitudinal slit arrangement.

15. The medical device according to claim 1, wherein the elastically deformable functional part is formed as a valve body.

16. The medical device according to claim 1, wherein the elastically deformable functional part has a head region, wherein the slit arrangement is formed along a transverse extent of the head region and extends centrally along a central longitudinal axis through the head region.

17. The medical device according to claim 1, wherein the medical device is a medical device for infusion therapy.

18. A method for sterilizing and/or for establishing sterilization resistance of the medical device according to claim 1,
wherein the method comprises the steps of:
a) coating or loading the slit wall faces of the slit arrangement with the composition; and
b) sterilizing the medical device or the elastically deformable functional part.

19. An elastically deformable functional part for a medical device, the elastically deformable functional part comprising a slit arrangement that is openable, wherein the slit arrangement widens or opens upon an elastic deformation of the elastically deformable functional part and recloses upon cessation of the elastic deformation, wherein slit wall faces of the slit arrangement are coated with a composition, wherein the composition comprises a silicone oil and a thickener.

20. A method for sterilizing and/or for establishing sterilization resistance of the elastically deformable functional part according to claim 19, wherein the method comprises the steps of:
a) coating or loading the slit wall faces of the slit arrangement with the composition; and
b) sterilizing the medical device or the elastically deformable functional part.
